(19) United States
(12) Patent Application Publication
(10) Pub. No.: US 2023/0256144 A1
(43) Pub. Date: Aug. 17, 2023

(54) Breast Pump Leak Guard

(71) Applicant: ASO LLC, Sarasota, FL (US)

(72) Inventors: Edmund A. Sinda, Sarasota, FL (US); Brooke A. Hagerty, Sarasota, FL (US); Mira Dosen, Sarasota, FL (US)

(21) Appl. No.: 17/671,943

(22) Filed: Feb. 15, 2022

(51) Int. Cl. A61M 1/06; A61F 13/60; A61F 13/53; A61F 13/45; A61F 13/511

(52) U.S. Cl. CPC A61M 1/062; A61F 13/60; A61F 13/53; A61F 13/45; A61F 13/51121; A61F 2013/15016

(57) ABSTRACT

A leak guard for use with a breast pump suction flange having a liquid extraction port through which liquid from a wearer's breast flows when the breast pump suction flange is worn by a wearer includes a flexible body defining an outer perimeter of the flexible body. The flexible body is configured to contact an exterior of the breast pump suction flange and substantially conform to an exterior contour thereof when worn. A liquid collection region defined by the flexible body within the outer perimeter is positioned beneath a bottom perimeter of the breast pump suction flange when the flexible body is worn to collect liquid leakage from the bottom perimeter.

### FIELD

This relates to the field of breast pumps and, more particularly to, capturing leakage from a breast pump suction flange.

### BACKGROUND

One of the major issues with breast pumping is that liquid can leak beyond the suction flange and onto one's skin during letdown, which is a sudden release of milk during breast pumping. Letdown occurs in waves multiple times throughout the pumping process until milk runs out. In the first letdown cycle, approximately one ounce of milk is released. As women differ in how much milk they produce and their breast and nipple size, the breast pump suction flange sometimes does not properly connect to the skin, allowing milk to leak out of the flange.

Another cause of leakage from the flange is when the suction is lost between the skin and the flange when the flange is detached after pumping is complete. The detachment can be abrupt and cause the milk to slide down the flange and onto one's clothing. For working mothers or mothers who pump in public, the milk that leaks onto clothing during pumping can cause embarrassing stains.

The breast pumping experience can become a hassle for mothers who are unable to identify a properly fitting flange. Moreover, breast pumps themselves are expensive and are often provided by insurance companies, which means many women do not have an option to experiment with different brands of breast pumps to find the best fit.

### BRIEF SUMMARY

What is needed is a device that can be used in conjunction with a conventional breast pump suction flange, which captures liquid leakage from the flange. A device that solves this problem is a leak guard that captures liquid leakage from the suction flange on a wearer's breast. The suction flange has a liquid extraction port through which liquid extracted from the breast flows. The leak guard includes a flexible body positionable on the flange in such a way that the body captures liquid leakage from the flange.

A first example of a leak guard for use with a breast pump suction flange having a liquid extraction port through which liquid from a wearer's breast flows when the breast pump suction flange is worn by a wearer includes a flexible body defining an outer perimeter of the flexible body. The flexible body is configured to contact an exterior of the breast pump suction flange and substantially conform to an exterior contour thereof when worn. A liquid collection region is defined by the flexible body within the outer perimeter and is positioned beneath a bottom perimeter of the breast pump suction flange when the flexible body is worn to collect liquid leakage from the bottom perimeter.

The first example of the leak guard may include one or more of the following features.

The outer perimeter may include adhesive.

The flexible body may include a liquid absorbent material capable of absorbing the liquid leakage.

The leak guard may include an adhesive on the flexible body beneath the liquid collection region for adhering the flexible body to skin of the wearer.

The flexible body may include a liquid impermeable material in the liquid collection region.

The flexible body may include a liquid impermeable material that contacts the exterior of the breast pump suction flange when the leak guard is worn.

The flexible body may emulate a disk shape and include a liquid extraction port receiving member that permits the liquid extraction port to extend therethrough. The flexible body may also include a liquid impermeable material in the liquid collection region and a liquid absorbent material capable of absorbing the liquid leakage.

The leak guard may include a breast pump and a breast pump suction flange.

A second example of a leak guard for use with a breast pump suction flange having a liquid extraction port through which liquid from a wearer's breast flows when the breast pump suction flange is worn by a wearer includes a body including liquid impermeable material configured to contact an exterior of the breast pump suction flange and remain in place during a breast pumping session. A liquid collection region defined by the body is positioned beneath the liquid extraction port when worn by the wearer for collecting liquid leakage from the breast pump suction flange. The leak guard also includes a liquid tight seal on the body beneath the liquid collection region for sealing liquid leakage in the liquid collection region.

The second example of the leak guard may include one or more of the following features.

The body may at least partially surround a radial axis of the liquid extraction port by 75 to 360 degrees.

The body may define an opening through which the liquid extraction port passes.

A tab may extend from the body that causes the body to release from the breast pump suction flange when pulled.

The liquid collection region may include a liquid absorbent material.

The liquid tight seal may include an adhesive on a bottom perimeter of the body that adheres the body to skin of the wearer when worn.

A bottom perimeter of the body may extend off the breast pump suction flange and contact skin of the wearer when worn.

The liquid collection region may be positioned beneath a bottom perimeter of the breast pump suction flange when the body is worn and a bottom perimeter of the body may extend off the breast pump suction flange and contact skin of the wearer when worn. The liquid tight seal may include an adhesive on the bottom perimeter of the body that adheres the body to skin of the wearer.

The leak guard may include a breast pump suction flange and a breast pump.

A third example of a leak guard for use with a breast pump suction flange having a liquid extraction port through which liquid from a wearer's breast flows when the breast pump suction flange is worn by a wearer includes a body including liquid impermeable material and an adhesive perimeter. A liquid collection region defined by the body is within the adhesive perimeter. The liquid collection region is positioned beneath a bottom perimeter of the breast pump suction flange when the body is worn. The adhesive perimeter adheres the body to the breast pump suction flange and skin of the wearer and forms a liquid tight seal.

The third example of the leak guard may include one or more of the following features.

The body may at least partially surrounds a radial axis of the liquid extraction port by 75 to 360 degrees.

The body may define an opening through which the liquid extraction port passes.

The leak guard may include a tab extending from the body that causes the body to de-adhere from the breast pump suction flange and skin when pulled.

The liquid collection region may include a liquid absorbent material.

The leak guard may include a breast pump suction flange and a breast pump in liquid communication with the breast pump suction flange.

An example of a method includes capturing liquid leakage from a breast pump suction flange attached to a wearer's breast while pumping milk from the wearer's breast by positioning a breast pump leak guard on the breast pump suction flange. The leak guard includes a flexible body defining an outer perimeter of the flexible body. The flexible body contacts an exterior of the breast pump suction flange and substantially conforms to an exterior contour thereof. A liquid collection region defined by the flexible body is within the outer perimeter. The liquid collection region is positioned beneath a bottom perimeter of the breast pump suction flange to collect liquid leakage from the bottom perimeter.

The method may include one or more of the following features.

The method may include removing and replacing a liquid absorbent material in the liquid collection region.

The outer perimeter may include adhesive.

The flexible body may include a liquid absorbent material capable of absorbing the liquid leakage.

The method may include adhering the flexible body beneath the liquid collection region to skin of the wearer.

The flexible body may include a liquid impermeable material in the liquid collection region.

The flexible body may include a liquid impermeable material that contacts the exterior of the breast pump suction flange.

The flexible body may emulate a disk shape and include a liquid extraction port receiving member that permits the liquid extraction port to extend therethrough. The flexible body may also include a liquid impermeable material in the liquid collection region and a liquid absorbent material capable of absorbing the liquid leakage.

The breast pump leak guard may be positioned on the breast pump suction flange without having any portion of the breast pump leak guard wrapping around a torso of the wearer.

### BRIEF DESCRIPTION OF THE DRAWINGS

FIG. 1 is a diagram of a conventional breast pump system.

FIG. 2 is a front view of an example of the breast pump leak guard.

FIG. 3 is a rear view thereof.

FIG. 4 is a side perspective view thereof.

FIG. 5 is a diagram illustrating a method of applying the leak guard over the breast pump suction flange.

FIG. 6 is a side view of the leak guard installed over the breast pump suction flange on a wearer's breast.

FIG. 7 is a front, or exterior, view thereof.

FIG. 8 is a side view of the leak guard installed over the flange on a wearer's breast, illustrating a method of removing the leak guard from the flange.

FIG. 9 is a front view of the breast pump suction flange with an angle indicating the degree to which the liquid extraction port is surrounded.

FIG. 10 is a front view of a second example of the leak guard.

FIG. 11 is a front view of a third example of the leak guard.

FIG. 12 is a front view of a fourth example of the leak guard.

FIG. 13 is a front view of a fifth example of the leak guard.

FIG. 14 is a front view of a sixth example of the leak guard.

FIG. 15 is a front view of a seventh example of the leak guard.

FIG. 16 is a side perspective view thereof.

FIG. 17 is a side view thereof installed over the flange on a wearer's breast.

### DETAILED DESCRIPTION OF EXAMPLES

This disclosure describes examples and aspects of a breast pump leak guard, but not all possible examples and aspects thereof. Where a particular feature is disclosed in the context of a particular aspect or example, that feature can also be used, to the extent possible, in combination with and/or in the context of other aspects and examples. The breast pump leak guard, may be embodied in many different forms and should not be construed as limited to only the examples and aspects described here.

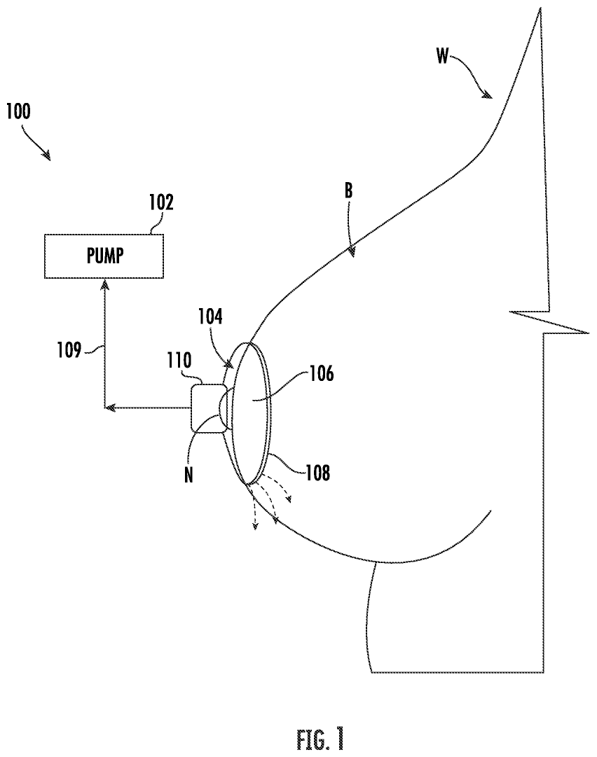

Referring to FIG. 1, a conventional breast pumping system 100 includes a pump 102 that applies suction to a suction flange 104 fitted tightly over the wearer's W breast B and extracts liquid from the breast B through a liquid conduit 109. The flange 104 includes a flange body 106 defining a flange perimeter 108 that encircles the wearer's nipple N. An extraction port 110 protruding from the flange body 106 proximal to the nipple N is attached to the conduit 109. In operation, the pump 102 applies suction via the conduit 109 through the extraction port 110 to the nipple N and liquid is extracted from the breast B. The problem with this construction is that liquid leakage, indicated by dashed-line arrows extending from the flange perimeter 108, often occurs.

It would be advantageous to have a leak guard that can be attached to a conventional breast pump suction flange 104 for capturing leakage therefrom. Such a breast pump leak guard, which is described here, includes a body positionable on the flange 104 about the extraction port 110 in such a way that the body captures liquid leakage from the flange 104.

Some examples of the breast pump leak guard are positionable on the flange 104 without having any portion of the breast pump leak guard wrapping around the wearer's torso, as would be the case with a garment. This allows the breast pump leak guard to be easily installed before pumping and removed thereafter without having the wearer change clothes.

Referring to FIGS. 2-4, a first example of the leak guard 200 includes a body 202 defining an extraction port receiving member 204, a liquid collection region 206, and an outer perimeter 208.

The body 202 is made of a substantially liquid impermeable material such as a plastic, rubber, rubber-like, or plastic-like material. In some examples, the material is a thin sheet of pliable substantially liquid impermeable plastic. The material may be flexible enough to conform to various breast shapes and may adapt to the changing breast size as pumping will shrink the breast back to normal size after engorgement. Flexibility of the material promotes comfort, which can also be achieved through the functionality of the leak guard 200. Examples of suitable materials include, but are not limited to, polyethylene, polyurethane, or the like.

The body 202 may be a thin sheet of the liquid impermeable material, such as a sheet that is not more than 2 mm thick or 1 mm thick.

The body 202 may be flexible enough to substantially conform to an exterior contour of the leak guard 200 when worn.

The liquid impermeable material may be breathable, meaning it may allow water vapor to pass therethrough.

The body 202 in the example shown in FIGS. 2-4 has an outer perimeter 208 defining the terminal edge of the body 202. A central region of the body 202 defines the extraction port receiving member 204 which, in this example, is a hole extending completely through the body 202 from an exterior side 210 to an interior side 212 of the body 202. The extraction port receiving member 204 is dimensioned to be able to receive the extraction port 110 of the breast pump suction flange 104 in such a way that the extraction port 110 passes through the extraction port receiving member 204.

The extraction port receiving member 204 is defined by an opening rim 214 that circumscribes the hole. The opening rim 214 may be expandable by including one or more slits 216 formed through the body 202 substantially perpendicular to the opening rim 214 to allow the extraction port receiving member 204 to expand around the extraction port 110 if necessary. Many conventional breast pump suction flange extraction ports come in various diameters from 14 mm to 36 mm to accommodate different sized nipples.

An upper section 215 of the body 202 includes a tab 218 that can be pulled by the wearer W for removal of the body 202 from the flange 104. The tab 218 may freely sit above the rest of the body 202 without having an adhesive on the interior side 212 to permit the W wearer to easily grab the tab 218 and pull when removing the leak guard 200 from the flange 104. In other examples, the tab 218 may include adhesive on the interior 212.

The body 202 includes a liquid tight seal 220 at least along a lower section of the perimeter 208. This may be achieved, for example, by having an adhesive along the interior side 212 adjacent the perimeter 208 or by using adhesive tape. The adhesive may be any adhesive suitable for use with materials that may contact human skin. Particular examples of adhesives include, but are not limited to, low trauma adhesives such as low trauma acrylic adhesive, low trauma silicone adhesive, and the like.

The liquid tight seal 220 is designed to prevent liquid captured by the leak guard 200 from leaking out the bottom of the leak guard 200. The liquid tight seal 220 enables the wearer to not have to lean forward during the pumping process. Since the wearer will be able to sit upright in a more-natural position, they will not only be more relaxed and comfortable, but the natural sitting position helps to speed up the pumping process and helps promote better milk flow.

The body 202 interior side 212 defines a liquid passage 222 through which liquid from the breast B can flow. The liquid passage 222 is in liquid communication with the liquid collection region 206.

The liquid collection region 206 is defined by the body 202 in the form of a pocket or the like in which leakage from the flange 104 collects. Accordingly, the liquid collection region 206 is loosely positioned over the skin when in use. If desired, the liquid collection region 206 may include a liquid absorbent material 226 configured to absorb the collected liquid in the liquid collection region 206 to prevent spillage when the leak guard 200 is removed from the flange 104.

The liquid absorbent material 226 may be made of liquid absorbent material such as cotton, polyester, wool, lyocell, or the like. The liquid absorbent material 226 may have many different shapes, but in some cases it is advantageous for the liquid absorbent material to have substantially a crescent shape as shown in FIGS. 2-4. With such a crescent shape, an upper edge 228 is curved to approximately follow the curvature of the bottom of the flange 104 and a lower edge 230 is curved to approximately follow the curvature of the bottom of the body 202.

The liquid absorbent material 226 is most advantageous in two situations: during pumping and during removal. During pumping, leakage may occur due to improper positioning of the flange 104, incorrect flange 104 size, or breast shape. Breast liquid leaks will be collected by the liquid absorbent material 226 and held there until disposal. The liquid absorbent material 226 may be permanently attached to or removable from the body interior 212. In some examples, the body 202 may be sold with several removable liquid absorbent material 226 units so that the body 202 can be reused and the used liquid absorbent material 226 thrown away.

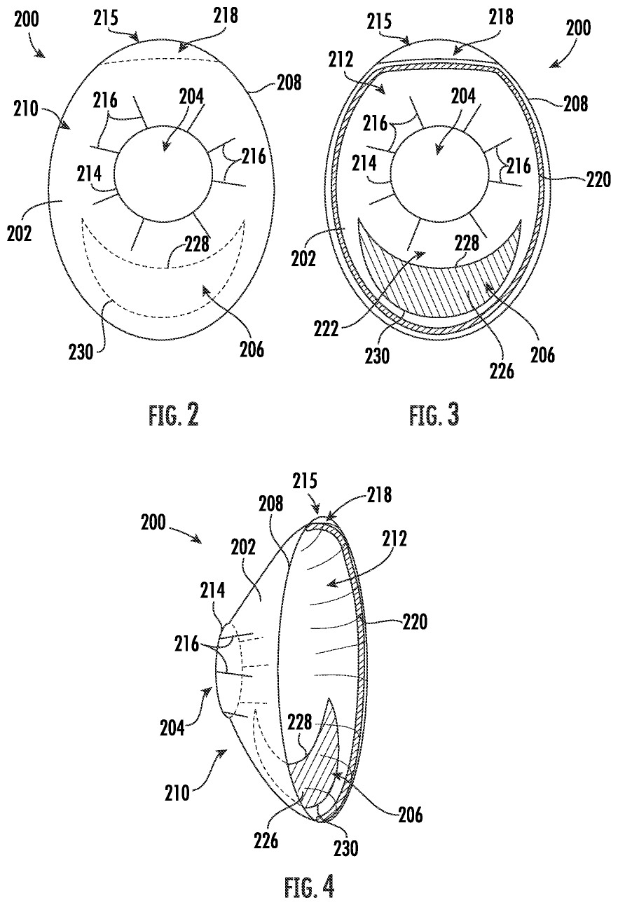

As shown in FIG. 4, the body 202 has a substantially frustoconical shape with the opening rim 214 forming one terminal end thereof and the perimeter 208 forming the opposite terminal end thereof. This frustoconical shape is designed to at least somewhat mimic the shape of the flange 104 to provide a tight substantially leak-proof fit over the flange 104.

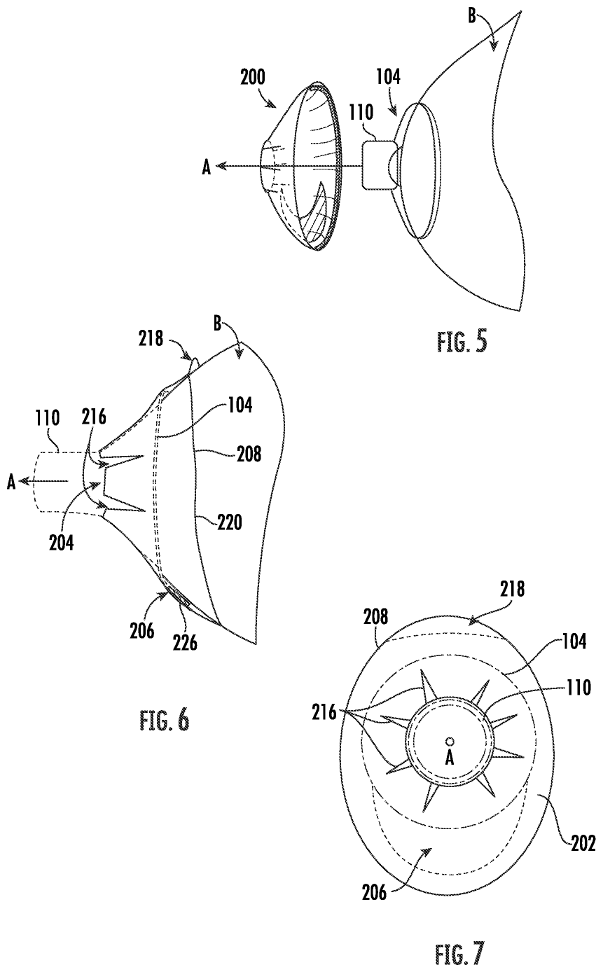

Referring to FIGS. 5-7, the leak guard of FIGS. 2-4, 200 is applied over the flange 104 by inserting the liquid extraction port 110 through the extraction port receiving member 204 in such a way that a radial axis A of the extraction port 110 passes through the extraction port receiving member 204. When the leak guard 200 is in the proper position, the body 202 extends off the flange 104 and the perimeter 208 contacts at least a lower portion of the of the breast B skin. The liquid tight seal 220 s formed by pressing the portion of the body 202 having adhesive against the breast B. The liquid collection region 206 and liquid absorbent material 226 are positioned beneath the bottom perimeter of the flange 104 to capture liquid leakage from the flange 104.

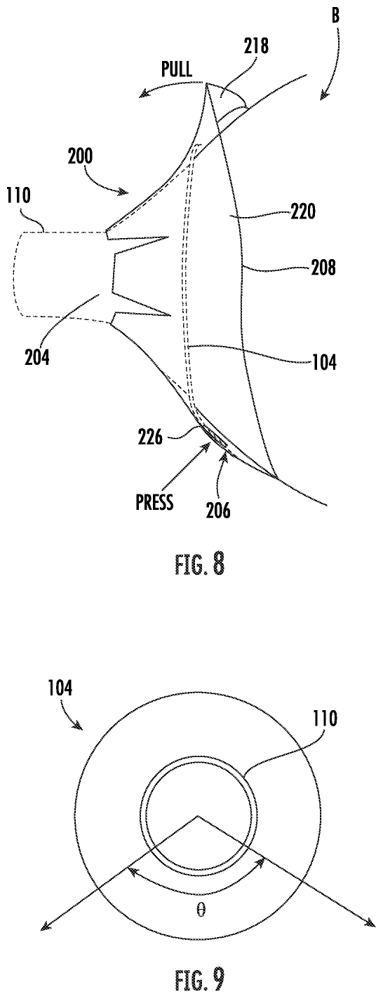

Referring to FIG. 8, the leak guard 200 is removable from the flange 104 by pulling downwardly and/or outwardly on the tab 218 away from the breast B while pressing on the liquid collection region 206 towards the breast B, which holds any leakage in the liquid collection region 206 in place during removal.

In the example of FIGS. 1-8, the body 202 surrounds the radial axis A of the liquid extraction port 110 by 360 degrees. Referring to FIG. 9, in other examples, the body 202 surrounds the radial axis A of the liquid extraction port 110 to a lesser degree, such as between an angle θ from 75 degrees to 360 degrees.

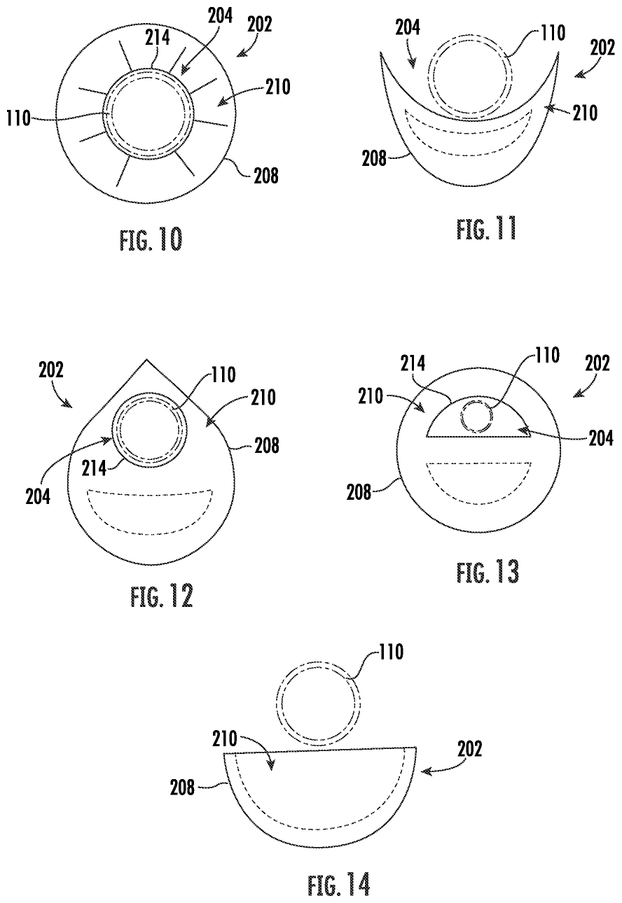

In FIGS. 10-14, alternate examples of the leak guard 200 are shown along with their location relative to the liquid extraction port 110.

In the example of FIG. 10, the body 202 surrounds the liquid extraction port 110 by 360 degrees and has a substantially circular perimeter 208 when viewed from the exterior side 210.

In the example of FIG. 11, the body 202 partially surrounds the liquid extraction port 110 and has a substantially crescent-shaped perimeter 208 when viewed from the exterior side 210.

In the example of FIG. 12, the body 202 surrounds the liquid extraction port 110 by 360 degrees and has a substantially teardrop-shaped perimeter 208 when viewed from the exterior side 210.

In the example of FIG. 13, the body 202 surrounds the liquid extraction port 110 by 360 degrees and has a substantially circular perimeter 208 when viewed from the exterior side 210. The extraction port receiving member 204 has a semicircle shape.

In the example of FIG. 14, the body 202 has a semicircular perimeter 208 when viewed from the exterior side 210. An upper portion of the perimeter 208 is straight and is positioned horizontally beneath and adjacent to the liquid extraction port 110.

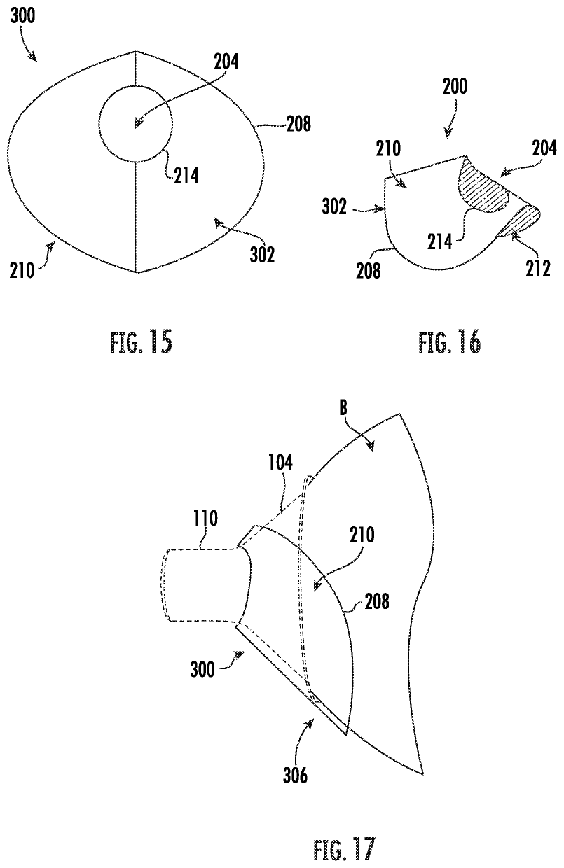

Referring to FIGS. 15-17, a seventh example of the breast pump leak guard 300 will now be described using the same reference numerals as above to refer to the corresponding features in the second example.

In the second example, the breast pump leak guard 300 includes a body 302 at least partially made of a liquid absorbent material. The body includes an outer perimeter 208 defining the terminal edge of the body 302. A central region of the body 302 defines the extraction port receiving member 204 which, in this example, is a hole through the body 302 extending completely through the body 302 from an exterior side 210 to an interior side 212 of the body 202. The extraction port receiving member 204 is dimensioned to be able to receive the extraction port 110 of the breast pump suction flange 104 in such a way that the extraction port 110 passes through the extraction port receiving member 204.

The extraction port receiving member 204 is defined by an opening rim 214 that circumscribes the hole. Although not shown in FIGS. 15-17, the opening rim 214 may be expandable as in the first example by including one or more slits formed through the body 302 substantially perpendicular to the opening rim 214 to allow the extraction port receiving member 204 to expand around the extraction port 110.

The body 302 is designed to approximately conform to the shape of the flange 104 by having a concave shape. The interior side 212 may include adhesive to keep the leak guard 300 in place while being worn. In certain other examples, the interior side 212 does not include an adhesive.

In use, the second example of the breast pump leak guard 300 is placed over the flange 140 such that the liquid extraction port 110 extends through the liquid extraction port receiving member 204. Because the body 300 has a concave shape, it substantially conforms to the curvature of the flange 104. When installed on the flange 104, a lower section 306 of the body 302 extends past a terminal bottom edge of the flange 104 and contacts the wearer's skin beneath the flange 104. This lower section 306 captures liquid leakage from around the bottom perimeter of the flange 104.

The body 302 may be made entirely of liquid absorbent material or partially of liquid absorbent material. The lower section 306 that extends off the flange and onto the wearer's skin should include the liquid absorbent material to be able to absorb leakage from the flange 104. The liquid absorbent material is a material capable of absorbing the leakage and retaining it therein. Such materials may include, for example, absorbent fabric materials such as cotton, polyester, polyester, lyocell, wool, or the like.

The body 302 may be a thin sheet of the liquid absorbent material, such as a sheet that is not more than 10 mm thick, 5 mm thick, 2 mm thick, or 1 mm thick.

The body 302 may be flexible enough to substantially conform to an exterior contour of the leak guard 200 when worn.

The body 302 may be formed with many different shapes. In the example of FIGS. 15-17, the body 302 emulates a disk shape. By emulating a disk shape, the body 302 resembles a disk whether the outer perimeter is circular or the outer perimeter 208 is not circular.

As used herein the term “substantially” means for the most part.

The breast pump leak guard may be modified in many different ways without departing from the scope of what is claimed. The scope of the claims is not limited to only the particular features and examples described above.

That which is claimed is:

1. A leak guard for use with a breast pump suction flange having a liquid extraction port through which liquid from a wearer's breast flows when the breast pump suction flange is worn by a wearer, the leak guard comprising:
a flexible body defining an outer perimeter of the flexible body, the flexible body being configured to contact an exterior of the breast pump suction flange and substantially conform to an exterior contour thereof when worn; and
a liquid collection region defined by the flexible body within the outer perimeter, the liquid collection region being positioned beneath a bottom perimeter of the breast pump suction flange when the flexible body is worn to collect liquid leakage from the bottom perimeter.

2. The leak guard of claim 1, wherein the outer perimeter includes adhesive.

3. The leak guard of claim 1, wherein the flexible body includes a liquid absorbent material capable of absorbing the liquid leakage.

4. The leak guard of claim 1, further comprising an adhesive on the flexible body beneath the liquid collection region for adhering the flexible body to skin of the wearer.

5. The leak guard of claim 1, wherein the flexible body includes a liquid impermeable material in the liquid collection region.

6. The leak guard of claim 1, wherein the flexible body includes a liquid impermeable material that contacts the exterior of the breast pump suction flange when the leak guard is worn.

7. The leak guard of claim 1, wherein the flexible body emulates a disk shape.

8. The leak guard of claim 1, wherein:
the flexible body includes a liquid extraction port receiving member that permits the liquid extraction port to extend therethrough;
the flexible body includes a liquid impermeable material in the liquid collection region; and
the flexible body includes a liquid absorbent material capable of absorbing the liquid leakage.

9. The leak guard of claim 1, further comprising a breast pump and a breast pump suction flange.

10. A leak guard for use with a breast pump suction flange having a liquid extraction port through which liquid from a wearer's breast flows when the breast pump suction flange is worn by a wearer, the leak guard comprising:
a body including liquid impermeable material, the body being configured to contact an exterior of the breast pump suction flange and remain in place during a breast pumping session;
a liquid collection region defined by the body, the liquid collection region being positioned beneath the liquid extraction port when worn by the wearer for collecting liquid leakage from the breast pump suction flange; and
a liquid tight seal on the body beneath the liquid collection region for sealing liquid leakage in the liquid collection region.

11. The leak guard of claim 10, wherein the body at least partially surrounds a radial axis of the liquid extraction port by 75 to 360 degrees.

12. The leak guard of claim 10, wherein the body defines an opening through which the liquid extraction port passes.

13. The leak guard of claim 10, further comprising a tab extending from the body that causes the body to release from the breast pump suction flange when pulled.

14. The leak guard of claim 10, wherein the liquid collection region includes a liquid absorbent material.

15. The leak guard of claim 10, wherein the liquid tight seal includes an adhesive on a bottom perimeter of the body that adheres the body to skin of the wearer when worn.

16. The leak guard of claim 10, wherein a bottom perimeter of the body extends off the breast pump suction flange and contacts skin of the wearer when worn.

17. The leak guard of claim 10, wherein
the liquid collection region is positioned beneath a bottom perimeter of the breast pump suction flange when the body is worn;
a bottom perimeter of the body extends off the breast pump suction flange and contacts skin of the wearer when worn; and
the liquid tight seal includes an adhesive on the bottom perimeter of the body that adheres the body to skin of the wearer.

18. The leak guard of claim 10, further comprising a breast pump suction flange and a breast pump.

19. A leak guard for use with a breast pump suction flange having a liquid extraction port through which liquid from a wearer's breast flows when the breast pump suction flange is worn by a wearer, the leak guard comprising:
a body including liquid impermeable material and an adhesive perimeter; and
a liquid collection region defined by the body within the adhesive perimeter, the liquid collection region being positioned beneath a bottom perimeter of the breast pump suction flange when the body is worn on the breast pump suction flange;
the adhesive perimeter adhering the body to the breast pump suction flange and skin of the wearer and forming a liquid tight seal.

20. The leak guard of claim 19, wherein the body at least partially surrounds a radial axis of the liquid extraction port by 75 to 360 degrees.

21. The leak guard of claim 19, wherein the body defines an opening through which the liquid extraction port passes.

22. The leak guard of claim 19, further comprising a tab extending from the body that causes the body to de-adhere from the breast pump suction flange and skin when pulled.

23. The leak guard of claim 19, wherein the liquid collection region includes a liquid absorbent material.

24. The leak guard of claim 19, further comprising a breast pump suction flange and a breast pump in liquid communication with the breast pump suction flange.

25. A method comprising:
capturing liquid leakage from a breast pump suction flange attached to a wearer's breast while pumping milk from the wearer's breast by positioning on the breast pump suction flange a breast pump leak guard comprising:
a flexible body defining an outer perimeter of the flexible body, the flexible body contacting an exterior of the breast pump suction flange and substantially conforming to an exterior contour thereof; and
a liquid collection region defined by the flexible body within the outer perimeter, the liquid collection region positioned beneath a bottom perimeter of the breast pump suction flange to collect liquid leakage from the bottom perimeter.

26. The method of claim 25, further comprising removing and replacing a liquid absorbent material in the liquid collection region.

27. The method of claim 25, wherein the outer perimeter includes adhesive.

28. The method of claim 25, wherein the flexible body includes a liquid absorbent material capable of absorbing the liquid leakage.

29. The method of claim 25, further comprising adhering the flexible body beneath the liquid collection region to skin of the wearer.

30. The method of claim 25, wherein the flexible body includes a liquid impermeable material in the liquid collection region.

31. The method of claim 25, wherein the flexible body includes a liquid impermeable material that contacts the exterior of the breast pump suction flange.

32. The method of claim 25, wherein the flexible body emulates a disk shape.

33. The method of claim 25, wherein:
the flexible body includes a liquid extraction port receiving member that permits the liquid extraction port to extend therethrough;
the flexible body includes a liquid impermeable material in the liquid collection region; and
the flexible body includes a liquid absorbent material capable of absorbing the liquid leakage.

34. The method of claim 25, wherein the breast pump leak guard is positioned on the breast pump suction flange without having any portion of the breast pump leak guard wrapping around a torso of the wearer.
